(19) United States
(12) Patent Application Publication
(10) Pub. No.: US 2022/0026439 A1
(43) Pub. Date: Jan. 27, 2022

(54) PARTICLE BASED SMALL MOLECULE-PROTEIN COMPLEX TRAP

(71) Applicants: Orionis Biosciences, Inc., Waltham, MA (US); Orionis Biosciences NV, Zwijnaarde (BE)

(72) Inventors: Nikolai KLEY, Waltham, MA (US); Samuel LIEVENS, Zwinjnaarde (BE); Riccardo SABATINI, Waltham, MA (US)

(21) Appl. No.: 17/415,248

(22) PCT Filed: Dec. 19, 2019
(86) PCT No.: PCT/US2019/067386

§ 371 Date: Jun. 17, 2021

Related U.S. Application Data
(60) Provisional application No. 62/782,524, filed on Dec. 20, 2018.

(51) Int. Cl. G01N 33/68; G01N 33/543

(52) U.S. Cl. CPC G01N 33/6845; G01N 2500/02; G01N 33/54313

(57) ABSTRACT

The present invention relates to the use of a particle, including a virus-like particle (VLP), for the discovery and analysis of protein-protein interactions that are modulated by small molecules.

### CROSS-REFERENCE TO RELATED APPLICATIONS

This application claims the benefit of U.S. Provisional Patent Application No. 62/782,524, filed Dec. 20, 2018; the entire contents of which are hereby incorporated by reference in their entirety.

### FIELD

The present invention relates to the use of a particle, including a virus-like particle (VLP), for the discovery and analysis of protein-protein interactions that are modulated by small molecules.

### SEQUENCE LISTING

The instant application contains a Sequence Listing which has been submitted in ASCII format via EFS-Web and is hereby incorporated by reference in its entirety. Said ASCII copy, created on Dec. 19, 2019, is named “ORN-056PC_Sequence_Listing” and is 4,312 bytes in size.

### BACKGROUND

A small molecule drug is a low molecular weight organic compound that can affect a biological process. Compared to larger biologics, small molecules are easier to manufacture and design. Further, small molecules provide a simpler chemical fingerprint which allows for more predictable biological effects. As such, small molecules make up more than ninety percent of the therapeutics on the market.

Nevertheless, there remains a significant gap between the number of promising small molecules identified through in vitro assays and those being able to achieve therapeutic efficacy with minimal side effects in vivo. In this regard, a better understanding of a small molecule's mechanism of action may facilitate in the discovery of more effective therapeutics. Specifically, an elucidation of how a small molecule may affect protein-protein interactions can aid in developing targeted agents with specific mechanisms of action. This not only includes elucidation of if (or how) small molecules may inhibit a protein-protein interaction but also, importantly, whether, how and in what way a small molecules may induce a protein-protein interaction. This is particularly important because protein-protein interactions are crucial components of virtually all cellular processes, including signal transduction, gene transcription, and cellular replication. Abnormal protein interactions are also associated with human diseases.

Accordingly, there is a need for novel methods for identifying and characterizing physiologically relevant small molecule-modulated protein-protein and small molecule/protein complex-protein interactions for drug discovery and therapeutic applications.

### SUMMARY

In one aspect, the present invention provides methods for identifying and characterizing protein-protein interactions that are modulated by small molecules. Such methods find utility in, for instance, drug discovery and translational medicine, with the objective of discovering mechanisms of action, and unknown targets, of small molecules. The present invention therefore enables anticipation or prediction of potential uses for small molecules which can induce or inhibit particular protein-protein interactions and thereby eliciting specific biological effects.

In various embodiments, the present invention provides a method for characterizing protein-protein interactions which are enhanced by, or mediated by, binding of a first interaction polypeptide with a small molecule. The method comprises: (1) expressing a construct comprising one or more particle-forming (or particle-associated) polypeptide fused to a first interaction polypeptide in a cell; (2) incubating the cell with one or more small molecules; (3) allowing the first interaction polypeptide to interact with the small molecule; (4) allowing a second interaction polypeptide to form a complex with a pre-formed complex comprising the first interaction polypeptide and the small molecule (5) isolating the particle; and (6) analyzing the protein-protein complex. In an illustrative embodiment, a GAG-CRBN fusion is interacted with an IMiD compounds and the resultant protein binding to the CRBN/IMiD is interrogated.

In various embodiments, the present invention also provides a method for characterizing protein-protein interactions which are inhibited by binding of a first interaction polypeptide with a small molecule. In various embodiments, the method comprises detecting the presence or absence of a protein-protein complex in the presence of absence of a small molecule and comparing the results (e.g. to detect the loss of a protein-protein complex in the presence of a small molecule). In various embodiments, the method comprises: (1) expressing a construct comprising one or more particle-forming (or particle-associated) polypeptide fused to a first interaction polypeptide in a cell; (2) allowing the first interaction polypeptide to interact with a second interaction polypeptide; (3) isolating the particle; (4) analyzing the protein-protein complex; (5) incubating the cell with one or more small molecules; (6) allowing the first interaction polypeptide to interact with the small molecule; (7) allowing the second interaction polypeptide to dissociate from the first interaction polypeptide; (8) isolating the particle; and (9) quantifying protein-protein complex (e.g. the analyzing a loss of an interaction polypeptide).

In the embodiments provided above, the methods can be utilized to identify a second interaction polypeptide which may be an endogenous protein or a recombinant protein.

In alternative embodiments, the present invention provides a method for characterizing protein-protein interactions comprising: (1) expressing a construct comprising one or more particle-forming (or particle-associated) polypeptide fused to a second interaction polypeptide in a cell; (2) incubating the cell with one or more small molecules; (3) allowing the small molecule to interact with a first interaction polypeptide and form a complex; (4) allowing the second interaction polypeptide to form a complex with the pre-formed complex comprising the first interaction polypeptide and the small molecule (5) isolating the particle; and (6) analyzing the protein-protein complex. In some embodiments, the small molecule may be applied as a library or pool of various known molecules and the analysis may also be of the small molecule.

In another embodiment, the present invention provides a method for characterizing protein-protein interactions comprising: (1) expressing a construct comprising one or more particle-forming (or particle-associated) polypeptide fused to a second interaction polypeptide in a cell; (2) allowing a first interaction polypeptide to interact with the second interaction polypeptide present in the cell (3) incubating the cell with one or more small molecules; (4) allowing the first interaction polypeptide to interact with the small molecule; (5) allowing the second interaction polypeptide to dissociate from the first interaction polypeptide; (6) isolating the particle; and (7) analyzing the protein-protein complex.

In the embodiments provided above, the methods can be utilized to identify a second interaction polypeptide which may be derived from a protein, cDNA, and/or open reading frame (ORF) library.

In another aspect, the present invention provides an artificial particle, for example, a virus-like particle (VLP), comprising a particle (e.g., VLP) forming polypeptide, a first interaction polypeptide, a second interaction polypeptide, and a small molecule. In some embodiments, the particle (e.g., VLP) forming polypeptide comprises the HIV p55 GAG protein or a fragment thereof.

In various embodiments, the first interaction polypeptide is known to interact with the small molecule and the second interaction polypeptide is unknown to interaction with the first interaction polypeptide and/or the small molecule.

### BRIEF DESCRIPTION OF THE DRAWINGS

FIG. 1A-C Identification of ASS1 as an endogenous molecular glue-induced CRBN neosubstrate using ViroTrap. Endogenous ASS1 was identified as an MiD-induced interactor of CRBN using a “protein trap” technology known as ViroTrap and described previously (Eyckerman, et al. “Trapping mammalian protein complexes in viral particles,” Nature Communications 7: 11416 (2016), the entire contents of which are herein incorporated by reference) and in more detail in Example 1. Binding of ASS1 to CRBN in response to lenalidomide (LEN; FIG. 1A), pomalidomide (POM; FIG. 1B) or CC-220 (FIG. 1C) was detected by identifying ASS1 tryptic peptides from a virus-like particle containing cellular CRBN recruited into such particle (including any associated protein or proteins) during the particle budding process from HEK293T cells. Accordingly, shown is a volcano plot of tryptic peptide identities for peptides isolated from CRBN-containing virus-like particles isolated with the ViroTrap procedure from cells exposed to lenalidomide, pomalidomide or CC-220 versus DMSO control vehicle (to identify lenalidomide-, pomalidomide- or CC-220-induced CRBN interactors). The plot shows p-values (Y-axis) versus fold difference (X-axis) of tryptic peptide signal between MiD-treated versus DMSO-treated, identifying ASS1 as a CRBN interactor induced by any of the three tested IMiDs, lenalidomide, pomalidomide and CC-220.

FIG. 2 Endogenous PPP3CA is detected as an FK506-induced FKBP12 binding protein. A similar ViroTrap approach as described in FIG. 1A-C was applied to identify FK506-induced interactors of FKBP12. Upon expression of a gag-FKBP12 fusion protein in HEK293T cells and differential treatment with either FK506 or DMSO as negative control, virus-like particles from FK506-treated cells were found to be enriched for the presence of PPP3CA, a calcineurin catalytic subunit and known binder of FKBP12 in the presence of FK506.

FIG. 3 Non-limiting schematic representation of an illustrative trapping method for identifying protein-protein interactions that are modulated by a small molecule of interest. CRBN=cereblon, DCAF=DDB1 and CUL4 Associated Factor, TF=transcription factor, EP=endogenous protein, RP=recombinant protein. The small molecule (SM) may bind and modulate the fused or unfused protein member of the protein-protein interaction.

### DETAILED DESCRIPTION

The present invention provides methods for identifying and characterizing protein-protein interactions that are either induced or inhibited by a small molecule. Particularly, methods of the invention utilize a particle, such as a virus-like particle (VLP), in which the ability of a small molecule-protein complex to interact with another protein is characterized.

In aspects, the present invention relates to a method for detecting an interaction between a small molecule/protein complex and another protein, comprising: (i) incubating a cell expressing a construct comprising one or more particle-forming (or particle-associated) polypeptides fused to a first interaction polypeptide with one or more small molecules to form a small molecule/protein complex; (ii) allowing a second interaction polypeptide to form a complex with the pre-formed small molecule/protein complex; and (iii) analyzing the small molecule/protein-protein complex in an isolated particle.

In aspects, the present invention relates to a method for detecting an interaction between a small molecule/protein complex and another protein, comprising: (i) expressing a construct comprising one or more particle-forming (or particle-associated) polypeptides fused to a first interaction polypeptide in a cell; (ii) incubating the cell or particle with one or more small molecules; (iii) allowing the first interaction polypeptide to interact with the small molecule and form a small molecule/protein complex; (iv) allowing a second interaction polypeptide to form a complex with the pre-formed small molecule/protein complex; (v) isolating the particle; and (vi) analyzing the small molecule/protein-protein complex.

In aspects, the present invention relates to a method for detecting a small molecule inhibition or reduction of a protein-protein interaction, comprising: (i) expressing a construct comprising one or more particle-forming (or particle-associated) polypeptide fused to a first interaction polypeptide in a cell which comprises second interaction polypeptide present in the cell to form a protein-protein interaction; (iii) incubating the cell or particle with one or more small molecules which interact with the first interaction polypeptide in a manner that causes the second interaction polypeptide to dissociate from the first interaction polypeptide; and (iii) analyzing the protein-protein complex in an isolated particle.

In aspects, the present invention relates to a method for detecting a small molecule inhibition or reduction of a protein-protein interaction, comprising: (i) expressing a construct comprising one or more particle-forming (or particle-associated) polypeptide fused to a first interaction polypeptide in a cell; (ii) allowing the first interaction polypeptide to interact with a second interaction polypeptide present in the cell; (iii) incubating the cell or particle with one or more small molecules; (iv) allowing the first interaction polypeptide to interact with the small molecule; (v) allowing the second interaction polypeptide to dissociate from the first interaction polypeptide; (vi) isolating the particle; and (vii) analyzing the protein-protein complex.

In aspects, the present invention relates to a method for detecting a protein-protein interaction, comprising (i) incubating a cell expressing a construct comprising one or more particle-forming (or particle-associated) polypeptides fused to a first interaction polypeptide with one or more small molecules to form a small molecule/protein complex; (ii) allowing a second interaction polypeptide to form a complex with the pre-formed small molecule/protein complex; and (iii) analyzing the protein-protein complex in an isolated particle.

In aspects, the present invention relates to a method for detecting a protein-protein interaction, comprising: (i) expressing a construct comprising one or more particle-forming (or particle-associated) polypeptide fused to a second interaction polypeptide in a cell; (ii) incubating the cell or particle with one or more small molecules; (iii) allowing the small molecule to interact with a first interaction polypeptide and form a complex; (iv) allowing the second interaction polypeptide to form a complex with the pre-formed complex comprising the first interaction polypeptide and the small molecule; (v) isolating the particle; and (vi) analyzing the protein-protein complex.

In aspects, the present invention relates to a method for detecting a protein-protein interaction, comprising (i) expressing a construct comprising one or more particle-forming (or particle-associated) polypeptide fused to a first interaction polypeptide in a cell which comprises second interaction polypeptide present in the cell to form a protein-protein interaction; (iii) incubating the cell or particle with one or more small molecules; (iii) incubating the cell or particle with one or more small molecules which interact with the first interaction polypeptide in a manner that causes the second interaction polypeptide to dissociate from the first interaction polypeptide; and (iii) quantifying a protein-protein complex in an isolated particle.

In aspects, the present invention relates to a method for detecting a protein-protein interaction, comprising: (i) expressing a construct comprising one or more particle-forming (or particle-associated) polypeptide fused to a second interaction polypeptide in a cell; (ii) allowing a first interaction polypeptide to interact with the second interaction polypeptide present in the cell; (iii) incubating the cell or particle with one or more small molecules; (iv) allowing the first interaction polypeptide to interact with the small molecule; (v) allowing the second interaction polypeptide to dissociate from the first interaction polypeptide; (vi) isolating the particle; and (vii) quantifying a protein-protein complex.

Particles

In various embodiments, the present invention provides a particle, comprising a particle-forming (or particle-associated) polypeptide, a first interaction polypeptide, a second interaction polypeptide, and a small molecule.

In various embodiments, the interaction between the first interaction polypeptide and the second interaction polypeptide is enhanced upon binding of the small molecule to the first interaction polypeptide. Without wishing to be bound by theory, it is believed that the small molecule may act like a molecular “glue” to promote the association of the first interaction polypeptide with the second interaction polypeptide, an association which otherwise would not occur or would only weakly occur. In an embodiment, the binding of the small molecule to the first interaction polypeptide induces a conformational change in the first interaction polypeptide, which is then able to bind to the second interaction polypeptide (e.g., with greater affinity). Such conformational change induction may be allosteric. In another embodiment, the binding of the small molecule to the first interaction polypeptide produces a novel interaction surface which can then be recognized by the second interaction polypeptide. In such an embodiment, both the small molecule and the first interaction polypeptide may physically interact with the second interaction polypeptide. In a further embodiment, the binding of the small molecule to the first interaction polypeptide can induce both a conformational change in the first interaction polypeptide as well as forming a novel interaction surface so as to induce or enhance the interaction with the second interaction polypeptide.

In alternative embodiments, the interaction between the first interaction polypeptide and the second interaction polypeptide is inhibited upon binding of the small molecule to the first interaction polypeptide. In some embodiments, the binding of the small molecule to the first interaction polypeptide induces a conformational change which results in a steric or allosteric loss of interaction between the first interaction polypeptide and the second interaction polypeptide.

As used herein, a “virus-like particle”, or “VLP” refers to a particle comprising at least a viral particle forming polypeptide. VLPs can be derived from numerous viruses. Examples of such particles have been described in the art and include, but are not limited to, particles derived from virus families including Parvoviridae (e.g., adeno-associated virus), Retroviridae (e.g., HIV), Flaviviridae (e.g., Hepatitis C virus), Orthomyxoviridae (e.g., Influenza virus), and Rhabdoviridae (e.g., vesicular stomatitis virus). In some embodiments, the particles comprise viral structural proteins, such as Envelope or Capsid, and result in the self-assembly of virus like particles. In various embodiments, the VLPs as described herein do not contain viral genetic material, so they are non-infectious. In some embodiments, the VLPs do not include viral DNA or RNA. In other embodiments, the VLPs as described herein contain viral genetic material, and are infectious. In some embodiments, the VLPs include viral DNA or RNA.

Virus-like particle forming polypeptide or VLP forming polypeptide is known in the art. Illustrative VLP forming polypeptides are described in US Patent Publication No. 2015/053355 and PCT Patent Publication No. WO 2016/150992, the entire disclosures of all of which are hereby incorporated by reference. In various embodiments, these terms refer to polypeptides or proteins that allow the assembly of viral particles, and, in some embodiments, the budding of said particles from a cell. A VLP forming polypeptide is sufficient to form a VLP, and in some embodiments, there are more than one (identical or non-identical) VLP forming polypeptides in a VLP.

In various embodiments, the VLP forming polypeptide may be any naturally occurring VLP forming protein known in the art, or a variant, derivative, or fragment thereof. In an exemplary embodiment, the VLP forming polypeptide comprises the HIV p55 GAG protein, or a variant, derivative, or fragment thereof.

In alternative embodiments, a viral structural protein other than HIV p55 GAG protein is utilized for forming the particle (e.g., VLP). In some embodiments, the viral structural protein may form a non-enveloped viral particle through multimerization. Any viral structural proteins known in the art and which are capable of forming a particle may be utilized for the present invention.

In various embodiments, the VLP forming polypeptide may be a modified form of a VLP forming protein known in the art. In various embodiments, the modifications do not inhibit formation of viral particles. In various embodiments, the VLP forming polypeptide may be a fragment of a VLP forming protein known in the art. In various embodiments, any modification or functional fragment of a VLP forming protein as contemplated herein is capable of forming virus-like particles that are capable of entrapping the small molecule-protein complex according to the invention.

In various embodiments, the present VLP forming polypeptide comprises an amino acid sequence having one or more amino acid mutations with respect to an amino acid sequence of any VLP forming protein known in the art. For example, the present VLP forming polypeptide may comprise an amino acid sequence having one, or two, or three, or four, or five, or six, or seen, or eight, or nine, or ten, or fifteen, twenty, thirty, forty, fifty, or one hundred amino acid mutations with respect to an amino acid sequence of any VLP forming protein known in the art. In some embodiments, the one or more amino acid mutations may be independently selected from substitutions, insertions, deletions, and truncations. In some embodiments, the modifications reduce the binding of the VLP forming polypeptide with host proteins so as to minimize false positives.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the present VLP forming polypeptide may be a derivative of any VLP forming protein known in the art. In some embodiments, the present VLP forming polypeptides include derivatives that are modified, i.e., by the covalent attachment of any type of molecule such that covalent attachment does not prevent the activity of the polypeptide. For example, but not by way of limitation, derivatives include those polypeptides that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc.

In various embodiments, the present invention provides a particle comprising a first interaction polypeptide which binds to a small molecule. The present invention further provides that the particle comprises a second interaction polypeptide whose interaction with the first interaction polypeptide is modulated by binding of the small molecule to the first interaction polypeptide. In various embodiments, the first and/or second interaction polypeptide may comprise any protein, protein subunit, or polypeptide.

In some embodiments, the first and/or second interaction polypeptide may comprise any protein known in the art.

In some embodiments, the first and/or second interaction polypeptide may be a modified form of a protein known in the art, including any variant, derivative, or fragment thereof. In some embodiments, the first and/or second interaction polypeptide may include any of the protein modifications as described elsewhere herein.

In some embodiments, the first and/or second interaction polypeptide may comprise a transcription factor protein. Exemplary transcription factor proteins include, but are not limited to, glucocorticoid receptor (GR), estrogen receptor (ER), androgen receptor (AR), MITF, p53, HIF1/2, MYC, MAX, MLL1, HOX family of proteins, ERG1, b-catenin, BCL6, and any other transcription factor protein known in the art.

In some embodiments, the first and/or second interaction polypeptide may comprise a cytoplasmic or nuclear signaling protein. Exemplary cytoplasmic and/or nuclear signaling proteins include, but are not limited to, RAS, RAF, GAP proteins, p85/PI3K, BRD4 or any other bromodomain protein, any PHD and chromo domain containing proteins, MCL1, BCL2, MDM2, IDH1/2, cereblon (CRBN), proteins involved with the homeostasis machinery including other DDB1 and CUL4-associated factors (DCAFs) and E3 ligase subunits, RNA splicing factors (e.g. SF3B and others), and any other cytoplasmic or nuclear signaling protein known in the art. In some embodiments, interaction polypeptides that interact with a complex of E7070 and DCAF15 are analyzed. Alternatively, in an embodiment, interaction polypeptides that interact with a complex of a small molecule ligand (e.g., dexamethasone, testosterone, estrogen etc.) with a transcription factor(s) (e.g., glucocorticoid receptor, androgen receptor, estrogen receptor) are analyzed.

In some embodiments, the first and/or second interaction polypeptide may comprise a membrane protein or a membrane associated protein. Exemplary membrane proteins or membrane associated proteins include, but are limited to, GPCRs, CFTR, RAS, ion channel proteins, MCL1, BCL2, HK1/hexokinase, IRE1, and any other membrane protein or membrane associated protein known in the art.

In embodiments, the first interaction polypeptide is an E3 ligase substrate binding subunit.

In embodiments, the E3 ligase substrate binding subunit is selected from cereblon (CRBN) and Von Hippel Lindau (VHL).

In some embodiments, the present methods allow for the identification of new interaction partners, e.g., substrates or neosubstrates of a protein that binds to a compound, the protein having a cage of three tryptophan residues that are capable of interacting with a glutarimide ring of the compound, e.g., via hydrogen binding. In some embodiments, the interaction partner, e.g., substrate and/or neosubstrate, has a surface β-hairpin loop, the surface β-hairpin loop optionally having an arrangement of three backbone hydrogen bond acceptors at the apex of a turn followed by a glycine residue. In some embodiments, the interaction partner, e.g., substrate and/or neosubstrate, has a degron motif (see, Meszaros, et al. Sci Signal 2017: 10, 470, the entire contents of which are incorporated by reference).

In some embodiments, the first interaction polypeptide is a protein having a cage of three tryptophan residues that are capable of interacting with a glutarimide ring of the compound (such as, immunomodulatory drugs or immunomodulatory imide drugs (IMiDs)), e.g., via hydrogen binding.

In some embodiments, the second interaction polypeptide, e.g., substrate and/or neosubstrate, has a surface β-hairpin loop, the surface β-hairpin loop optionally having an arrangement of three backbone hydrogen bond acceptors at the apex of a turn followed by a glycine residue. In some embodiments, the second interaction polypeptide, e.g., substrate and/or neosubstrate, has a degron motif (see, Meszaros, et al. Sci Signal 2017: 10, 470, the entire contents of which are incorporated by reference).

In embodiments, the E3 ligase substrate binding subunit is associated with a scaffold protein. The scaffold protein is selected from, in various embodiments, damaged DNA binding protein 1 (DDB1), Cullin-4A (CUL4A), regulator of cullins 1 (ROC1), SKIP1, SKP1 interacting partner (SKIP2), Beta-transducin repeats-containing protein (β-TrCP), Double minute 4 protein (MDM4), X-Linked Inhibitor of Apoptosis (XIAP), DDB1 And CUL4 Associated Factor 15 (DCAF15), and WD Repeat Domain 12 (WDR12).

In embodiments, the small molecule is a molecular glue as described herein and known in the art.

In some embodiments, the present methods are applicable to the use of VHL as a E3 ligase substrate binding first interaction polypeptide protein. VHL is, similarly to CRBN, the substrate binding subunit of an E3 ligase. Accordingly all embodiments relating to an E3 ligase as first interaction polypeptide are equally applicable to VHL as first interaction polypeptide.

In embodiments, the present methods are applicable to the use FKBP12 protein or a member of this family (e.g. FK506 binding proteins), instead of an E3 ligase, as first interaction polypeptide (accordingly all embodiments relating to E3 ligase as first interaction polypeptide are equally applicable to FKBP12 protein or a member of this family as first interaction polypeptide).

In embodiments, the FKBP is selected from FKBP12, FKBP38 and FKBP52.

FKBP12 is known to bind the immunosuppressant molecule tacrolimus (FK506). In embodiments, the small molecule is FK506 or a derivative, analog, enantiomer or a mixture of enantiomers, or a pharmaceutically acceptable salt, solvate, hydrate, co-crystal, clathrate, or polymorph thereof. In embodiments, the small molecule is FK506 (tacrolimus), rapamycin (sirolimus), and cyclosporin A (CsA) or a derivative or analog thereof or a compound that binds to the same FKBP binding site as the FK506 (tacrolimus), rapamycin (sirolimus), and cyclosporin A (CsA) or a derivative or analog thereof.

In various embodiments, the identities of the first interaction polypeptide and the small molecule are known so as to identify a second interaction polypeptide whose interaction with the first interaction polypeptide is modulated by binding of the small molecule to the first interaction polypeptide. In some embodiments, the second interaction polypeptide is an endogenous protein or a recombinant protein. In some embodiments, the second interaction polypeptide may be derived from protein, cDNA, and/or open reading frame (ORF) libraries.

In various embodiments, the identities of the first and second interaction polypeptides are known so as to identify a small molecule whose interaction with the first interaction polypeptide can modulate the interaction between the first and second interaction polypeptides. In such embodiment, the small molecule may be derived from libraries. For example, the small molecule may be derived from compound libraries.

As used herein, “small molecules” are low molecular weight organic compounds, having a molecular weight of about 10,000 Daltons or less. In various embodiments, the small molecule may have a molecular weight of about 10,000, about 9,000, about 8,000, about 7,000, about 6,000, about 5,000, about 4,000, about 3,000, about 2,000, about 1,000, about 900, about 800, about 700, about 600, about 500, about 400, about 300, about 200, or about 100 Daltons or less. In various embodiments, the small molecule may be naturally occurring or synthetic.

In various embodiments, the small molecule is in its “native” form—i.e. without coupling of the small molecule to a moiety used for the assay (e.g. another small molecule, a “purification handle,” a bead, and the like). Accordingly, in some embodiments, the lack of small molecule coupling prevents or reduces false negative or false positive small molecule-protein interactions related to artifacts from the couple moiety.

In embodiments, the small molecule is a single small molecule, and not a first and second small molecule covalently linked to one another.

In embodiments, the small molecule binds to an interaction polypeptide, such as the first interaction polypeptide, without the need for a recruiting element, e.g. another small molecule linked to the small molecule.

In embodiments, the small molecule does not function as a bait to attract an interacting polypeptide. In embodiments, the small molecule binds to an interaction polypeptide, such as the first interaction polypeptide and that complex serves as a bait for a second interaction polypeptide. In embodiments, the small molecule induces a conformational change in an interaction polypeptide, such as the first interaction polypeptide, and the conformationally changed first interaction polypeptide interacts with the second interaction polypeptide. In embodiments, the small molecule induces an allosteric modification of the protein surface of the interaction polypeptide, such as the first interaction polypeptide (e.g. without limitation inducing exposure of a hydrophobic surface of the interaction polypeptide, such as the first interaction polypeptide), to allow interaction with the second interaction polypeptide. In embodiments, the small molecule does not interact with the second interaction polypeptide.

In various embodiments, the small molecules can be any organic small molecule, small molecule compounds made with combinatorial synthetic chemistry techniques (e.g., commercial small compound libraries), small molecule compounds with scaffolds as developed for inhibitors/modulators of kinases, GPCRs, proteases, and other enzymes, small molecule diversity oriented synthesis (DOS) compounds, small molecule compounds with one or more chiral centers, natural product small molecules (e.g., derived from plants, microorganisms, and other sources), and small compounds derived from fragment libraries (e.g., less than 250 Daltons in molecular weight).

In various embodiments, the small molecule is an immunomodulatory compound, e.g. which inhibits LPS induced monocyte TNF-α, IL-1β, IL-12, IL-6, MIP-1α, MCP-1, GM-CSF, G-CSF, and/or COX-2 production.

In embodiments, the small molecule is a molecular glue.

In some embodiments, the small molecule is an immunomodulatory agent. In some embodiments, the small molecule is a derivative of glutamic acid that comprises a glutarimide ring, optionally, and a phthalimide ring. In some embodiments, the phthalimide ring is chemically modified. In some embodiments, the derivative of glutamic acid can be a synthetic derivative having the properties in accordance with embodiments of the present disclosure. In some embodiments, the small molecule is a member of the class of compounds known as immunomodulatory drugs or immunomodulatory imide drugs (IMiDs). In embodiments, the small molecule contains an IMiD-like glutarimide ring, but otherwise differs in chemical structure and binds to the same small molecule binding pocket as a glutarimide-IMiD in CRBN (the IMiD binding pocket in CRBN). In embodiments, the small molecule does not contain a glutarimide ring and can bind CRBN in the IMiD pocket. In embodiments, the small molecule binds CRBN, but not in the IMiD pocket. In embodiments, the IMiD pocket is contained within the CULT (cereblon domain of unknown activity, binding cellular ligands and thalidomide) domain of CRBN, see PDB entries 4TZ4, 5FQD, 5HXB, 5V3O, 6H0F, and 6H0G and PLoS Comput Biol. 2015 January; 11(1): e1004023., each of which are incorporated by reference in their entireties.

In some embodiments, the small molecule is thalidomide, lenalidomide, pomalidomide, CC-220, CC-122, CC-885, or a derivative, analog, enantiomer or a mixture of enantiomers, or a pharmaceutically acceptable salt, solvate, hydrate, co-crystal, clathrate, or polymorph thereof.

In some embodiments, the small molecule is avadomide, endomide, iberdomide, lenalidomide, mitindomide, pomalidomide, and thalidomide, or a derivative, analog, enantiomer or a mixture of enantiomers, or a pharmaceutically acceptable salt, solvate, hydrate, co-crystal, clathrate, or polymorph thereof.

In some embodiments, the small molecule is an immunomodulatory drug (IMiD). Illustrative IMiDs include, but are not limited to, thalidomide and analogs or derivatives thereof including lenalidomide, pomalidomide, and CC-220.

Exemplary immunomodulatory small molecules include, but are not limited to, N-{[2-(2,6-dioxo(3-piperidyl)-1,3-dioxoisoindolin-4-yl]methyl}cyclopropylcarboxamide; 3-[2-(2,6-dioxopiperidin-3-yl)-1,3-dioxo-2,3-dihydro-1H-isoindol-4-ylmethyl]-1,1-dimethylurea; (−)-3-(3,4-dimethoxyphenyl)-3-(1-oxo-1,3-dihydroisoindol-2-yl)-propionamide; (+)-3-(3,4-dimethoxyphenyl)-3-(1-oxo-1,3-dihydroisoindol-2-yl)-propionamide; (−)-{2-[1-(3-ethoxy-4-methoxyphenyl)-2-methylsulfonylethyl]-4-acetylaminoisoindoline-1,3-dione}; (+)-{2-[1-(3-ethoxy-4-methoxyphenyl)-2-methylsulfonylethyl]-4-acetylaminoisoindoline-1,3-dione}; difluoromethoxy SeICIDs; 1-phthalimido-1-(3,4-diethoxyphenyl)ethane; 3-(3,4-dimethoxyphenyl)-3-(3,5-dimethoxyphenyl)acrylonitrile; 1-oxo-2-(2,6-dioxopiperidin-3-yl)-4-aminoisoindoline; 1,3-dioxo-2-(2,6-dioxopiperidin-3-yl)-4-aminoisoindoline; 4-amino-2-(3-methyl-2,6-dioxopiperidine-3-yl)isoindole-1,3-dione; 3-(3-acetoamidophthalimido)-3-(3-ethoxy-4-methoxyphenyl)-N-hydroxypropionamide; 1-oxo-2-(2,6-dioxopiperidin-3-yl)-4-methylisoindoline; cyclopropyl-N-{2-[(1S)-1-(3-ethoxy-4-methoxyphenyl)-2-(methylsulfonyl)ethyl]-3-oxoisoindoline-4-yl}carboxamide; substituted 2-(3-hydroxy-2,6-dioxopiperidin-5-yl)isoindoline; N-[2-(2,6-dioxopiperidin-3-yl)-1,3-dioxo-2,3-dihydro-1H-isoindol-5-ylmethyl]-4-trifluoromethoxybenzamide; (S)-4-chloro-N-((2-(3-methyl-2,6-dioxopiperidin-3-yl)-1,3-dioxoisoindolin-5-yl)methyl)benzamide; pyridine-2-carboxylic acid [2-[(3S)-3-methyl-2,6-dioxopiperidin-3-yl]-1,3-dioxo-2,3-dihydro-1H-isoindol-5-ylmethyl]amide; (S)—N-((2-(3-methyl-2,6-dioxopiperidin-3-yl)-1,3-dioxoisoindolin-5-yl)methyl)-4-(trifluoromethyl)benzamide; and 3-(2,5-dimethyl-4-oxo-4H-quinazolin-3-yl)piperidine-2,6-dione.

Exemplary immunomodulatory small molecules include, but are not limited to, cyano and carboxy derivatives of substituted styrenes, including, but not limited to, those disclosed in U.S. Pat. No. 5,929,117; 1-oxo-2-(2,6-dioxo-3-fluoropiperidin-3-yl)isoindolines and 1,3-dioxo-2-(2,6-dioxo-3-fluoropiperidine-3-yl)isoindolines, including, but not limited to, those described in U.S. Pat. Nos. 5,874,448 and 5,955,476; tetra substituted 2-(2,6-dioxopiperdin-3-yl)-1-oxoisoindolines, including, but not limited to, those described in U.S. Pat. No. 5,798,368; 1-oxo- and 1,3-dioxo-2-(2,6-dioxopiperidin-3-yl)isoindolines (e.g., 4-methyl derivatives of thalidomide); substituted 2-(2,6-dioxopiperidin-3-yl)phthalimides, and substituted 2-(2,6-dioxopiperidin-3-yl)-1-oxoisoindoles, including, but not limited to, those disclosed in U.S. Pat. Nos. 5,635,517, 6,281,230, 6,316,471, 6,403,613, 6,476,052, and 6,555,554; 1-oxo- and 1,3-dioxoisoindolines substituted in the 4- or 5-position of the indoline ring (e.g., 4-(4-amino-1,3-dioxoisoindoline-2-yl)-4-carbamoylbutanoic acid), including, but not limited to, those described in U.S. Pat. No. 6,380,239; isoindoline-1-one and isoindoline-1,3-dione substituted in the 2-position of the indoline ring with 2,6-dioxo-3-hydroxypiperidin-5-yl (e.g., 2-(2,6-dioxo-3-hydroxy-5-fluoropiperidin-5-yl)-4-aminoisoindolin-1-one), including, but not limited to, those described in U.S. Pat. No. 6,458,810; non-polypeptide cyclic amides, including, but not limited to, those disclosed in U.S. Pat. Nos. 5,698,579 and 5,877,200; and isoindole-imide compounds, including, but not limited to, those described in U.S. Pat. App. Pub. Nos. 2003/0045552 and 2003/0096841, and International Pub. No. WO 02/059106. The disclosure of each of the patents and patent application publications identified herein is incorporated herein by reference in its entirety.

In various embodiments, the immunomodulatory compound (e.g. IMID) binds to and modulates CRBN and the present methods identify further proteins that interact with the modulated CRBN. In various embodiments, the polypeptides that interact with a complex of E7070 and DCAF15 can be analyzed.

In various embodiments, use of linkers is contemplated herein. For example, in some embodiments, the particle (e.g., VLP) forming polypeptide is linked to the first interaction polypeptide through protein fusion (i.e. the VLP forming polypeptide is fused to the first interaction polypeptide). In some embodiments, the particle (e.g., VLP) forming polypeptide is fused to the first interaction polypeptide through a linker. In some embodiments, the particle (e.g., VLP) forming polypeptide is linked to the second interaction polypeptide through protein fusion (i.e. the VLP forming polypeptide is fused to the second interaction polypeptide). In some embodiments, the particle (e.g., VLP) forming polypeptide is fused to the second interaction polypeptide through a linker. In various embodiments, the presence of the linker allows the first or second interaction polypeptide to maintain its natural physiological protein conformation.

The invention contemplates the use of a variety of linker sequences. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et al., (2000), Protein Eng. 13(5):309-312, the entire contents of which are hereby incorporated by reference.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is a polypeptide. In some embodiments, the linker is greater than about 100 amino acids long. For example, the linker may be greater than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In various embodiments, the linker is substantially comprised of glycine and serine residues (e.g. about 30%, or about 40%, or about 50%, or about 60%, or about 70%, or about 80%, or about 90%, or about 95%, or about 97% glycines and serines). For example, in some embodiments, the linker is (Gly₄Ser)ₙ (SEQ ID NO: 1), where n is from about 1 to about 8, e.g. 1, 2, 3, 4, 5, 6, 7, or 8. In an embodiment, the linker sequence is GGSGGSGGGGSGGGGS (SEQ ID NO: 2). Additional illustrative linkers include, but are not limited to, linkers having the sequence LE, GGGGS (SEQ ID NO: 3), (GGGGS)ₙ (n=1-4) (SEQ ID NO: 4), (Gly)₈ (SEQ ID NO: 5), (Gly)₆ (SEQ ID NO: 6), (EAAAK)ₙ (n=1-3) (SEQ ID NO: 7), A(EAAAK)ₙA (n=2-5) (SEQ ID NO: 8), AEAAAKEAAAKA (SEQ ID NO: 9), A(EAAAK)₄ALEA(EAAAK)₄A (SEQ ID NO: 10), PAPAP (SEQ ID NO: 11), KESGSVSSEQLAQFRSLD (SEQ ID NO: 12), EGKSSGSGSESKST (SEQ ID NO: 13), GSAGSAAGSGEF (SEQ ID NO: 14), and (XP)ₙ, with X designating any amino acid, e.g., Ala, Lys, or Glu. In various embodiments, the linker is GGS.

In some embodiments, the linker is one or more of GGGSE (SEQ ID NO: 15), GSESG (SEQ ID NO: 16), GSEGS (SEQ ID NO: 17), GEGGSGEGSSGEGSSSEGGGSEGGGSEGGGSEGGS (SEQ ID NO: 18), and a linker of randomly placed G, S, and E every 4 amino acid intervals.

In some embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). In various embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence Cys-Pro-Pro-Cys which, when dimerized by disulfide bond formation, results in a cyclic octapeptide believed to act as a pivot, thus conferring flexibility. In various embodiments, the present linker comprises, one, or two, or three of the upper hinge region, the core region, and the lower hinge region of any antibody (e.g., of IgG, IgA, IgD, and IgE, inclusive of subclasses (e.g. IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin.

In various embodiments, the linker of the present invention comprises one or more glycosylation sites. In various embodiments, the linker is a hinge-CH2-CH3 domain of a human IgG4 antibody.

If desired, the present chimeric protein can be linked to an antibody Fc region, comprising one or both of C_{H2} and C_{H3} domains, and optionally a hinge region.

In some embodiments, the linker is a synthetic linker such as PEG.

In various embodiments, the particle (e.g., VLP) forming polypeptide, or a multitude thereof, forms a particle, including a hollow particle, in which the small molecule and the first and/or second interaction polypeptides are entrapped. In particular embodiments, the first and/or second interaction polypeptides are anchored within the particle, thereby ensuring the capturing of the complex formed by the small molecule and the first and/or second interaction polypeptides inside the particle (e.g., VLP). In an embodiment, the anchoring of the first and/or second interaction polypeptides to the particle structure is directed by the particle (e.g. VLP) forming polypeptide.

In various embodiments, the interaction between the small molecule and the first interaction polypeptide can be covalent or non-covalent, and can be direct or indirect. In an embodiment, the interaction between the small molecule and the first interaction polypeptide is indirect and is mediated through an accessory protein that itself interacts with the small molecule.

In various embodiments, the present invention provides a construct comprising a particle (e.g. VLP) forming polypeptide linked to a first or second interaction polypeptide. In some embodiments, the present invention provides a construct comprising only the first or second interaction polypeptide without the particle forming polypeptide. The construct can be generated using recombinant DNA techniques known in the art. For example, DNA sequences encoding the construct of the invention (e.g., DNA sequences encoding the particle (e.g., VLP) forming polypeptide linked to a first or second interaction polypeptide) can be chemically synthesized using methods known in the art. Synthetic DNA sequences can be ligated to other appropriate nucleotide sequences, including, e.g., expression control sequences, to produce gene expression constructs encoding the desired construct. Accordingly, in various embodiments, the present invention provides for isolated nucleic acids comprising a nucleotide sequence encoding the various constructs of the invention (e.g., a construct comprising a particle (e.g., VLP) forming polypeptide linked to a first or second interaction polypeptide).

Applications

The particles, including VLPs, provided herein are useful for identifying and characterizing protein-protein interactions that are either induced or inhibited by a small molecule, particularly in native and/or physiological conditions. Accordingly, in various embodiments, the use of a particle (e.g., VLP), as described herein, is provided for the identification and characterization of protein-protein interactions.

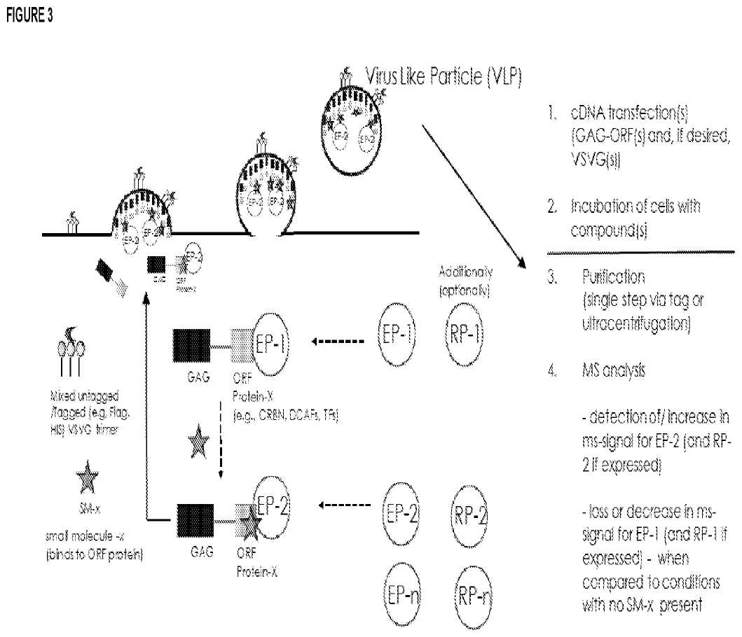

In various embodiments, the present particles (e.g., VLPs) are used for the discovery and identification of endogenous proteins or recombinant proteins whose interaction with a protein of interest is modulated by a known small molecule. A schematic representation of an illustrative method is provided in FIG. 3.

Specifically, in such embodiments, the present methods may be utilized for characterizing protein-protein interactions which are enhanced by binding of a first interaction polypeptide with a small molecule. The method comprises: (1) expressing a construct comprising one or more particle-forming (or particle-associated) polypeptide fused to a first interaction polypeptide in a cell; (2) incubating the cell with one or more small molecules; (3) allowing the first interaction polypeptide to interact with the small molecule and form a complex; (4) allowing a second interaction polypeptide to form a complex with the pre-formed complex comprising the first interaction polypeptide and the small molecule (5) isolating the particle; and (6) analyzing the protein-protein complex.

In some embodiments, the present methods may be utilized for characterizing protein-protein interactions which are inhibited by binding of a first interaction polypeptide with a small molecule. The method comprises: (1) expressing a construct comprising one or more particle-forming (or particle-associated) polypeptide fused to a first interaction polypeptide in a cell; (2) allowing the first interaction polypeptide to interact with a second interaction polypeptide present in the cell (3) incubating the cell with one or more small molecules; (4) allowing the first interaction polypeptide to interact with the small molecule and form a complex; (5) allowing the second interaction polypeptide to dissociate from the first interaction polypeptide; (6) isolating the particle; and (7) quantifying the protein-protein complex. In various embodiments, quantification of a protein-protein complex in the presence or absence of small molecule is undertaken.

In various embodiments, the methods described above identify a second interaction polypeptide which can be an endogenous protein or a recombinant protein whose interaction with the first interaction polypeptide is modulated by binding of the small molecule to the first interaction polypeptide. In some embodiments, the first interaction polypeptide is a protein of interest whose identity is known.

In various embodiments, the present particles (e.g., VLPs) are used for the discovery and identification of proteins whose interaction with an endogenous or recombinant protein of interest is modulated by a known small molecule. In such embodiments, the protein may be derived from a protein, cDNA, or ORF library. In various embodiments, the protein, cDNA, or ORF library may represent the entire genome of an organism.

Specifically, in such embodiments, the present methods may comprise: (1) expressing a construct comprising one or more particle-forming (or particle-associated) polypeptide fused to a second interaction polypeptide in a cell; (2) incubating the cell with one or more small molecules; (3) allowing the small molecule to interact with a first interaction polypeptide and form a complex; (4) allowing the second interaction polypeptide to form a complex with the pre-formed complex comprising the first interaction polypeptide and the small molecule (5) isolating the particle; and (6) analyzing the protein-protein complex.

In other embodiments, the present methods may comprise: (1) expressing a construct comprising one or more particle-forming (or particle-associated) polypeptide fused to a second interaction polypeptide in a cell; (2) allowing a first interaction polypeptide to interact with the second interaction polypeptide present in the cell (3) incubating the cell with one or more small molecules; (4) allowing the first interaction polypeptide to interact with the small molecule and form a complex; (5) allowing the second interaction polypeptide to dissociate from the first interaction polypeptide; (6) isolating the particle; and (7) analyzing the protein-protein complex.

In various embodiments, the methods described above identifies a second interaction polypeptide which is derived from a protein, cDNA, and/or open reading frame (ORF) library and whose interaction with the first interaction polypeptide is modulated by binding of the small molecule to the first interaction polypeptide. In some embodiments, the first interaction polypeptide may be an endogenous protein or a recombinant protein whose identity is known.

In various embodiments, the present particles (e.g., VLPs) are used for the discovery and identification of small molecules capable of modulating the protein-protein interaction of two known proteins. In such embodiments, the identities of the first and second interaction polypeptide are known so as to identify a small molecule whose interaction with the first interaction polypeptide can modulate the interaction between the first and second interaction polypeptides. In such embodiment, the small molecule may be derived from libraries. For example, the small molecule may be derived from compound libraries.

In some embodiments, instead of incubating the cells with one or more small molecules, the particles themselves (e.g., VLPs) are incubated with the small molecule(s). In such embodiments, the particles are permeable. Alternatively, the particles can be permeabilized using a permeabilization agent known in the art (e.g., an ionophore) or by any other chemical or mechanical means. In some embodiments, methods of incubating particles themselves (e.g., VLPs) with the small molecules are particularly useful for assessing interactions of the first interaction polypeptide with small molecules which are toxic to cells. Accordingly, the toxic small molecules can be screened at much higher concentrations when being incubated with particles rather than cells.

In various embodiments, the present methods comprise a step of expressing a construct comprising a particles (e.g., VLP) forming polypeptide linked to a first or second interaction polypeptide (or any other constructs described herein) in a cell. Any cell may be utilized in the present invention, including, but not limited to, plant cells, bacterial cells, viral cells, fungal cells, insect cells, and mammalian cells. In an embodiment, the cell is a mammalian cell such as a mammalian cell line. Exemplary mammalian cell lines include, but are not limited to, COS-1 or COS-7 (monkey kidney-derived), L-929 (murine fibroblast-derived), C127 (murine mammary tumor-derived), 3T3 (murine fibroblast-derived), CHO (Chinese hamster ovary-derived; including DHFR CHO (Urlaub et al., Proc. Natl. Acad. Sci. USA 77:4216 (1980)), HeLa (human cervical cancer-derived), BHK (hamster kidney fibroblast-derived, e.g., BHK21), PER.C6 (human embryonic retinal cells), and HEK-293 (human embryonic kidney-derived) cell lines and variants thereof. In some embodiments, the mammalian cell lines are monkey kidney CVI line transformed by SV40 (COS-7); human embryonic kidney line (293 or 293 cells as described, e.g., in Graham et al., Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CVI); African green monkey kidney cells (VERO-76); human cervical carcinoma cells (HELA); canine kidney cells (MDCK; buffalo rat liver cells (BRL 3A); human lung cells (W138); human liver cells (Hep G2); HKB11 cells (a somatic cell fusion between human kidney and human B cells as described in example, U.S. Pat. No. 6,136,599); mouse mammary tumor cells (MMT 060562); TRI cells (as described, e.g., in Mather et al., Annals N Y. Acad. Sci. 383:44-68 (1982)); MRC 5 cells; FRhL-2 cells, NIH3T3 cell, Jurkat, and FS4 cells. In some embodiments, useful mammalian cell lines include myeloma cell lines such as Y0, NS0, Sp2/0, NS1, Ag8, and P3U1. In various embodiments, useful cell lines include those with stably integrated Flp Recombination Target (FRT) sites so as to allow generation of stable cell lines using the Flp-In system.

In some embodiments, a cDNA encoding the construct comprising a particle (e.g., VLP) forming polypeptide linked to a first or second interaction polypeptide (or any other constructs described herein) is transiently transfected into cells.

In other embodiments, cell lines are generated which stably express a construct comprising a particle (e.g., VLP) forming polypeptide linked to a first or second interaction polypeptide (or any other constructs described herein). Generation of stable cell lines through homologous recombination is known in the art. For example, cell lines engineered to stably express a particle (e.g., VLP) forming polypeptide linked to a first or second interaction polypeptide can be generated using the Flp-In System. In some embodiments, stable cell lines are generated in which a single copy of the particle (e.g., VLP) forming polypeptide linked to an interaction polypeptide (or any other constructs described herein) is expressed from a locus of choice. In various embodiments, various cell lines can then be interrogated separately or analyzed as pools.

In yet further embodiments, expression of the construct comprising a particle (e.g. VLP) forming polypeptide linked to a first or second interaction polypeptide (or any other constructs described herein) may be regulated such as by an inducible system. For example, the construct may be inducibly expressed using a doxycycline inducible system. Methods for achieving transient, inducible, and/or stable expression of a construct in cells are known.

In some embodiments, the cells may additionally express the vesicular stomatitis virus glycoprotein (VSV-G), a transmembrane protein typically used for pseudotyping lentiviral vectors. In some embodiments, the VSV-G may be tagged (e.g., FLAG-tag). In some embodiments, the cell lines may be transiently, inducible, or stably transfected with a cDNA that encodes VSV-G or a tagged version of VSV-G. In an embodiment, the cell expressing VSV-G or a tagged version of VSV-G is subsequently fused with other cells.

In various embodiments, the present methods comprise a step of isolating particles (e.g., VLPs) for characterization. In some embodiments, isolating the particles (e.g., VLPs) can be done by means of a purification tag. In an embodiment, a tagged version (e.g. FLAG-tag) of VSV-G can be expressed in the cells to allow one-step purification of the particles (e.g., VLPs). In some embodiments, isolation is done by affinity chromatography or a similar method using an antibody directed against the tag (e.g. M2 anti-FLAG antibody). In some embodiments, antibodies directed against VSV-G itself or other molecules present on the surface can be used to enrich particles (e.g., VLPs). Other exemplary methods of particles (e.g., VLPs) enrichment include ultracentrifugation of supernatant containing particles (e.g., VLPs), gradient centrifugation of supernatant containing particles (e.g., VLPs), or precipitation of the particles (e.g., VLPs) from supernatant using precipitating agents. These methods require a centrifugation step to pellet particles (e.g., VLPs). These pellets can be re-dissolved and processed for analysis. Filtration kits to enrich and purify particles are known in the art. Particles (e.g. VLPs) may also be purified, enriched or isolated using flow-based methods (e.g., FACS or microfluidic-based separations methods).

In various embodiments, the identification of a first or second interaction polypeptide as well as a small molecule is a mass spectrometry based analysis. For example, the protein-protein interactions as described herein are monitored in the presence and absence of small molecules. In some embodiments, a small molecule may be identified by mass spectrometry. In some embodiments, the first and/or second interaction polypeptide may be identified by peptide fingerprint mass spectrometry.

The methods of the invention provide various advantages compared to other known methods for assessing protein-protein interactions. In contrast to in vitro methods, the present invention obviates the need for protein purification and assay development. Further, the present methods utilize cells which can express proteins that cannot be generated in vitro (e.g., full length proteins, proteins with acidic character, membrane associated proteins).

In various embodiments, the present methods allow for the detection of protein-protein interactions under physiologically relevant conditions. In some embodiments, the present methods allow for an analysis of proteins which may be post-translationally modified in cells, or even presented in complex with an endogenous protein. Accordingly, the present methods allow for an analysis of interactions that may dependent upon such modifications of the protein which cannot be identified with other in vitro protein interaction assays.

A co-purification technique called “Virotrap” was described in WO 2013/174999 and allows for evaluating protein-protein interactions in their physiological environment. However, Virotrap does not identify previously unknown protein-protein interactions, in which one of the proteins is modulated by a small molecule. In contrast, the present methods allow for scalable screening of unknown protein-protein interactions utilizing, for example, protein, cDNA, or ORF libraries that, for example, represent an entire genome and/or small molecule compound libraries.

Further still, unlike the Virotrap method or other hybrid systems for assaying protein interactions, the present method does not involve modifications of the small molecule (e.g., as a hybrid ligand). In the present methods, small molecule interaction with an interaction polypeptide of interest is identified directly through its recruitment into the VLP and subsequent small molecule mass-spectrometry based identification. Such direct recruitment of the small molecule-target protein complex into a VLP has several advantages. For example, no purification of the small molecule-target protein complex is required. In contrast, in vitro affinity-based purification methods usually associated with signal loss. In addition, lower affinity interactions are more likely to be identified using the present methods as no purification of the protein complex is required. Further, the present methods are associated with far less background noise as no solid phase purification materials are required (proteins often stick to such in vitro surfaces).

In embodiments, the present methods identify new interactions partners of an E3 ligase substrate binding subunit, such as selected from cereblon (CRBN) and Von Hippel Lindau (VHL). In embodiments, the present methods identify new interactions partners of an FKBP.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

### EXAMPLES

### Example 1: ViroTrap-Based Discovery of Endogenous Argininosuccinate Synthase 1 (ASS1) as a Substrate/Neosubstrate Recruited to Cereblon by Molecular Glues

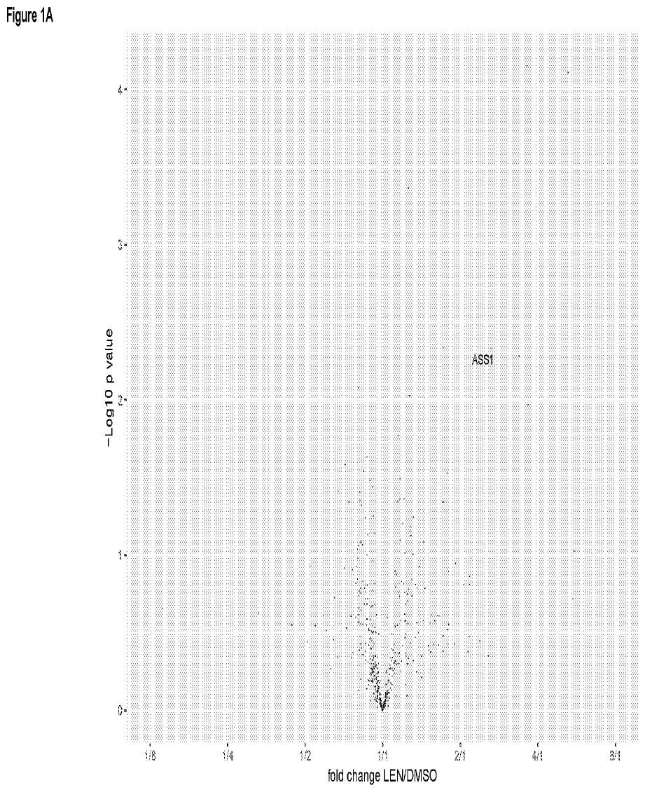

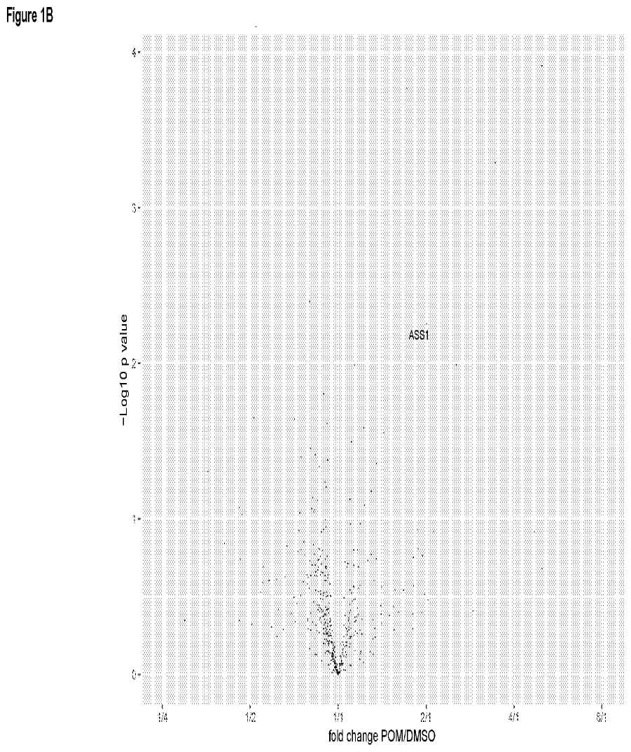

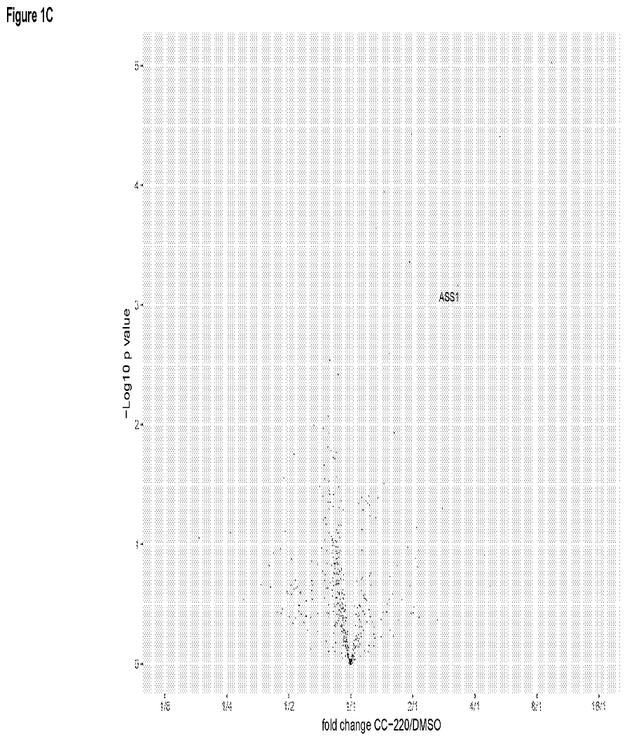

In this study we evaluated the use of ViroTrap to identify molecular glue-induced endogenous (neo)substrates of CRBN in HEK293T cells. For this purpose, we used a previously described method known as ViroTrap (Eyckerman, et al. “Trapping mammalian protein complexes in viral particles,” Nature Communications 7: 11416 (2016); Titeca, et al. “Analyzing trapped protein complexes by Virotrap and SFINX.” Nature Protocols 12.5 (2017): 881). With this method, CRBN is expressed as a fusion protein with the viral protein HIV-GAG. HIV-GAG, when expressed in cells, such as HEK293T, is capable of triggering the formation of virus-like particles that bud off the cells. In the particle, the HIV-GAG protein is oriented towards the center of the particle. When fused to CRBN, CRBN is displayed also in the internal core of the particle. In the event the GAG-CRBN fusion interacts with proteins inside the cells, such proteins are dragged/trapped along with GAG-CRBN into the virus-like particles. In this manner, endogenous proteins that interact with CRBN, in presence or absence of a CRBN-ligand, can be identified. This is achieved by lysis of the particle and a standard mass-spectrometry analysis of tryptic digest of the sample. Subtractive analysis will then identify proteins that are specifically associated with the particle in response to a CRBN-ligand such as a molecular glue. Using this method, we discovered that any of the molecular glues lenalidomide, pomalidomide or CC-220 induced the association of endogenous ASS1 with CRBN, i.e., ASS1 tryptic digests identified ASS1 as a protein recruited into the viral particle in dependence of lenalidomide, pomalidomide or CC-220. In short, HEK293T cells were co-transfected with a gag-CRBN bait expression plasmid and a Flag-VSV-G encoding plasmid (for particle purification from the cell medium), and incubated for 24 hours. Next, cells were treated with 10 μM lenalidomide, 10 μM pomalidomide, 1 μM CC-220 or DMSO as negative control. Twenty-four hours after compound addition, virus-like particles (VLPs) were isolated from the cell supernatant using anti-Flag coated magnetobeads. After elution from the beads using Flag peptide, purified VLPs were lysed, and the protein content was digested overnight with trypsin. The peptide samples were analyzed through LC-MS/MS and label-free quantification data (LFQ intensities) from triplicate samples was processed using the MaxQuant software package and a volcano plot showing p-value (resulting from a two-sided t-test corrected for multiple testing; FDR 0.05%) versus LFQ intensity fold change (FIG. 1A-C) was generated with the Perseus tool. ASS1 was identified among the top hits exhibiting a high signal ratio for the molecule glue versus DMSO control samples and a low p-value. The values plotted in FIG. 1A-C are presented in Table 1.

### Example 2: ViroTrap Identifies FK506-Dependent Binding of Calcineurin to FKBP12

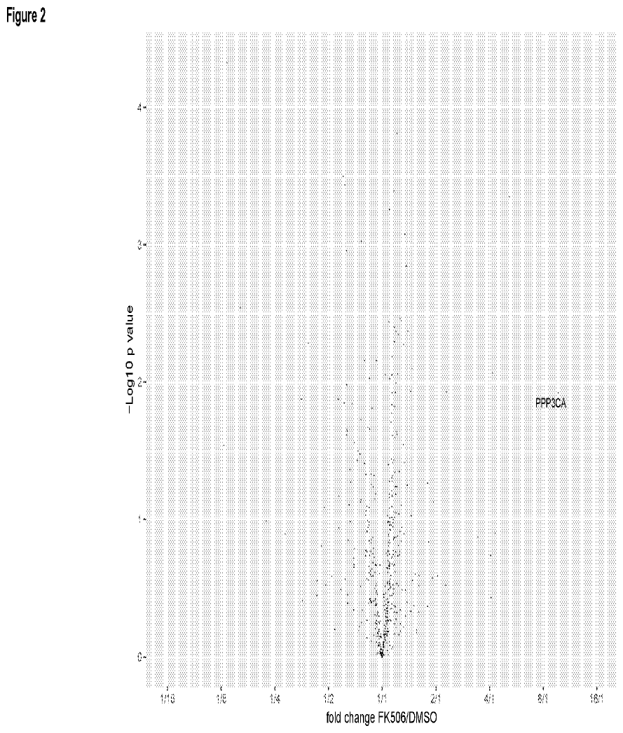

In this Example 2 we applied a similar approach as in Example 1, now using a gag-FKBP12 fusion protein which is expressed in HEK293T cells. Subsequently, cells were differentially treated with 1 μM FK506 (tacrolimus), and VLPs were isolated, processed and analyzed as described in Example 1. The data shown in FIG. 2 indicates that PPP3CA, a catalytic subunit of calcineurin and a known FK506-induced binding partner of FKBP12, was identified. The values plotted in FIG. 2 are presented in Table 1.

Table 1 summarizes the p-value and fold change plotted in the volcano plots shown in FIGS. 1A-C and FIG. 2.

| Bait | | Target | | Fold |
|---|---|---|---|---|
| protein | Compound | protein | p-value | change |
| CRBN | Lenalidomide (10 μM) | ASS1 | 4.65E−03 | 2.63 |
| CRBN | Pomalidomide (10 μM) | ASS1 | 5.58E−03 | 2.01 |
| CRBN | CC-220 (1 μM) | ASS1 | 6.85E−04 | 3.31 |
| FKBP12 | FK506 (1 μM) | PPP3CA | 1.19E−02 | 9.71 |

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method for detecting an interaction between a small molecule/protein complex and another protein, comprising:
(i) expressing a construct comprising one or more particle-forming (or particle-associated) polypeptides fused to a first interaction polypeptide in a cell;
(ii) incubating the cell or particle with one or more small molecules;
(iii) allowing the first interaction polypeptide to interact with the small molecule and form a small molecule/protein complex;
(iv) allowing a second interaction polypeptide to form a complex with the pre-formed small molecule/protein complex;
(v) isolating the particle; and
(vi) analyzing the small molecule/protein-protein complex.

2. A method for detecting a small molecule inhibition or reduction of a protein-protein interaction, comprising:
(i) expressing a construct comprising one or more particle-forming (or particle-associated) polypeptide fused to a first interaction polypeptide in a cell;
(ii) allowing the first interaction polypeptide to interact with a second interaction polypeptide present in the cell;
(iii) incubating the cell or particle with one or more small molecules;
(iv) allowing the first interaction polypeptide to interact with the small molecule;
(v) allowing the second interaction polypeptide to dissociate from the first interaction polypeptide;
(vi) isolating the particle; and
(vii) analyzing the protein-protein complex.

3. The method of claim 1 or 2, wherein the second interaction polypeptide is an endogenous protein or a recombinant protein whose identity or interaction capacity is unknown.

4. A method for detecting a protein-protein interaction, comprising:
(i) expressing a construct comprising one or more particle-forming (or particle-associated) polypeptide fused to a second interaction polypeptide in a cell;
(ii) incubating the cell or particle with one or more small molecules;
(iii) allowing the small molecule to interact with a first interaction polypeptide and form a complex;
(iv) allowing the second interaction polypeptide to form a complex with the pre-formed complex comprising the first interaction polypeptide and the small molecule;
(v) isolating the particle; and
(vi) analyzing the protein-protein complex.

5. A method for detecting a protein-protein interaction, comprising:
(i) expressing a construct comprising one or more particle-forming (or particle-associated) polypeptide fused to a second interaction polypeptide in a cell;
(ii) allowing a first interaction polypeptide to interact with the second interaction polypeptide present in the cell;
(iii) incubating the cell or particle with one or more small molecules;
(iv) allowing the first interaction polypeptide to interact with the small molecule;
(v) allowing the second interaction polypeptide to dissociate from the first interaction polypeptide;
(vi) isolating the particle; and
(vii) quantifying a protein-protein complex.

6. The method of claim 4 or 5, wherein the second interaction polypeptide is derived from a protein, cDNA, and/or open reading frame (ORF) library and whose identity or interaction capacity is unknown.

7. The method of any one of the above claims, wherein the particle is a virus-like particle (VLP).

8. The method of any one of the above claims, wherein the particle forming polypeptide comprises a p55 GAG protein or a variant, derivative, or fragment thereof.

9. The method of any one of the above claims, wherein the particle further comprises the spike glycoprotein of the vesicular stomatitis virus (VSV-G).

10. The method of any one of the above claims, wherein the particle lacks the spike glycoprotein of the vesicular stomatitis virus (VSV-G).

11. The method of claim 9 or 10, wherein the VSV-G is tagged or derivatized.

12. The method of any one of the above claims, wherein the construct further comprises one or more linkers.

13. The method of any of the above claims, wherein the isolating is carried out by affinity chromatography, centrifugation, or any tag-based method.

14. The method of any of the above claims, wherein the analyzing is carried out by mass spectrometry.

15. The method of claim 14, wherein the analyzing comprises comparing mass spectrometry fingerprints in the presence and absence of the small molecule.

16. The method of any of the above claims, wherein the first and/or second interaction polypeptide is a protein, cDNA, and/or open reading frame (ORF) library.

17. The method of any of the above claims, wherein the small molecule is a small molecule library.

18. The method of any of the above claims, wherein the small molecule is not coupled to another moiety, including another small molecule, a purification handle, a bead, and the like.

19. The method of any of the above claims, wherein the small molecule does not interact with the second interaction polypeptide.

20. The method of any of the above claims, wherein the first interaction polypeptide is an E3 ligase substrate binding subunit.

21. The method of claim 20, wherein the E3 ligase substrate binding subunit is selected from cereblon (CRBN) and Von Hippel Lindau (VHL).

22. The method of claim 21, wherein the E3 ligase substrate binding subunit is associated with a scaffold protein.

23. The method of claim 22 wherein the scaffold protein is selected from damaged DNA binding protein 1 (DDB1), Cullin-4A (CUL4A), regulator of cullins 1 (ROC1), SKIP1, SKP1 interacting partner (SKIP2), Beta-transducin repeats-containing protein (β-TrCP), Double minute 4 protein (MDM4), X-Linked Inhibitor of Apoptosis (XIAP), DDB1 And CUL4 Associated Factor 15 (DCAF15), and WD Repeat Domain 12 (WDR12).

24. The method of claim 20 or 21, wherein the small molecule is a molecular glue.

25. The method of any of claims 1-19, wherein the first interaction polypeptide is an FK506 binding protein (FKBP).

26. The method of claim 25, wherein the FKBP is selected from FKBP12, FKBP38 and FKBP52.

27. The method of claim 25 or 26, wherein the small molecule is FK506 (tacrolimus), rapamycin (sirolimus), and cyclosporin A (CsA) or a derivative or analog thereof or a compound that binds to the same FKBP binding site as the FK506 (tacrolimus), rapamycin (sirolimus), and cyclosporin A (CsA) or a derivative or analog thereof.


SEQUENCE LISTING (18 sequences)

SEQ ID NO: 1 - protein, 5 aa, Artificial Sequence
  Synthetic Sequence
GGGGS

SEQ ID NO: 2 - protein, 16 aa, Artificial Sequence
  Synthetic Sequence
GGSGGSGGGGSGGGGS

SEQ ID NO: 3 - protein, 5 aa, Artificial Sequence
  Synthetic Sequence
GGGGS

SEQ ID NO: 4 - protein, 5 aa, Artificial Sequence
  Synthetic Sequence
GGGGS

SEQ ID NO: 5 - protein, 8 aa, Artificial Sequence
  Synthetic Sequence
GGGGGGGG

SEQ ID NO: 6 - protein, 6 aa, Artificial Sequence
  Synthetic Sequence
GGGGGG

SEQ ID NO: 7 - protein, 5 aa, Artificial Sequence
  Synthetic Sequence
EAAAK

SEQ ID NO: 8 - protein, 12 aa, Artificial Sequence
  Synthetic Sequence
AEAAAKEAAAKA

SEQ ID NO: 9 - protein, 12 aa, Artificial Sequence
  Synthetic Sequence
AEAAAKEAAAKA

SEQ ID NO: 10 - protein, 46 aa, Artificial Sequence
  Synthetic Sequence
AEAAAKEAAAKEAAAKEAAAKALEAEAAAKEAAAKEAAAKEAAAKA

SEQ ID NO: 11 - protein, 5 aa, Artificial Sequence
  Synthetic Sequence
PAPAP

SEQ ID NO: 12 - protein, 18 aa, Artificial Sequence
  Synthetic Sequence
KESGSVSSEQLAQFRSLD

SEQ ID NO: 13 - protein, 14 aa, Artificial Sequence
  Synthetic Sequence
EGKSSGSGSESKST

SEQ ID NO: 14 - protein, 12 aa, Artificial Sequence
  Synthetic Sequence
GSAGSAAGSGEF

SEQ ID NO: 15 - protein, 5 aa, Artificial Sequence
  Synthetic Sequence
GGGSE

SEQ ID NO: 16 - protein, 5 aa, Artificial Sequence
  Synthetic Sequence
GSESG

SEQ ID NO: 17 - protein, 5 aa, Artificial Sequence
  Synthetic Sequence
GSEGS

SEQ ID NO: 18 - protein, 35 aa, Artificial Sequence
  Synthetic Sequence
GEGGSGEGSSGEGSSSEGGGSEGGGSEGGGSEGGS